(19) United States
(12) Patent Application Publication
(10) Pub. No.: US 2022/0015710 A1
(43) Pub. Date: Jan. 20, 2022

(54) SYSTEMS AND METHODS FOR PATIENT POSITIONING FOR IMAGING ACQUISITION

(71) Applicant: GE Precision Healthcare LLC, Wauwatosa, WI (US)

(72) Inventors: Chelsey A Lewis, Waukesha, WI (US); Michelle DeLong Samalik, Waukesha, WI (US); Franco Rupcich, Waukesha, WI (US)

(21) Appl. No.: 16/932,182

(22) Filed: Jul. 17, 2020

(51) Int. Cl. A61B 5/00; A61B 5/055; A61B 5/107

(52) U.S. Cl. CPC A61B 5/704; A61B 5/706; A61B 5/0555; A61B 5/7405; A61B 5/7264; A61B 5/107; A61B 5/0077

(57) ABSTRACT

Systems and methods for self-positioning patients on a table of an imaging system are described herein. In some examples, the method includes detecting a patient on a table proximate to a system. The method can also include providing a position indicator to the patient using one or more lights of the system, a camera of the system, a removable sheet, a display device of the system, or a combination thereof, and providing a modified position indicator in response to input received by the system.

### FIELD

Embodiments of the subject matter disclosed herein relate to non-invasive diagnostic imaging, and more particularly, to patient positioning for medical imaging.

### BACKGROUND

Non-invasive imaging technologies allow images of the internal structures of a patient or object to be obtained without performing an invasive procedure on the patient or object. In particular, technologies such as computed tomography (CT), among others, use various physical principles, such as the differential transmission of x-rays through the target volume, to acquire image data and to construct tomographic images (e.g., three-dimensional representations of the interior of the human body or of other imaged structures).

### SUMMARY

This summary introduces concepts that are described in more detail in the detailed description. It should not be used to identify essential features of the claimed subject matter, nor to limit the scope of the claimed subject matter.

In an aspect, a system for positioning a patient can include a processor that can detect a patient proximate to the system and detect an anatomical scan range of the patient for acquisition in a medical image. The processor can also determine that a first patient position prevents acquiring the medical image within the anatomical scan range and generate a position indicator to provide to the patient, the position indicator representing a second patient position that allows the system to acquire the medical image within the anatomical scan range of the patient.

In some examples, a system can be an x-ray imaging system, a computed tomography (CT) imaging system, a magnetic resonance imaging (MRI) system, a positron emission tomography (PET) imaging system, a single-photon emission computed tomography (SPECT) imaging system, and combinations thereof. In some aspects, the patient resides on a table proximate to the system and the position indicator comprises one or more lights displayed by the system using the table or using a display device of the system. In some examples, the one or more lights comprise at least a first light displaying a first color representing the first patient position or a second color representing the second patient position.

In some examples, the processor can project the position indicator onto the table and the patient indicator comprises a configuration image representing the second patient position that enables the system to acquire the medical image within the anatomical scan range. In some aspects, the system can project the position indicator from within a bore hole of the system, wherein the position indicator comprises one or more projected lights representing the second position. In some examples, the processor can capture one or more camera images of the patient with a camera and determine the first patient position based on the one or more camera images. The processor can also execute a machine learning technique to identify the first patient position. In some examples, the processor can detect a size of the patient and adjust the position indicator based on the size of the patient. The system can include a camera to project the position indicator onto the table. In some examples, the position indicator can include an audio message that provides a distance for the patient to move in one or more directions until the system detects that the patient is in the second patient position.

In an aspect, the system can include a material coupled to the table, wherein the material provides the position indicator, the position indicator comprising an outline of the second patient position. In some examples, the table is configured in a vertical position proximate the system or the table is configured in a horizontal position proximate the system. In one aspect, the table comprises one or more lights that provide the patient indicator. In some examples, the processor can detect a physical characteristic of the patient, the physical characteristic comprising a height of the patient and modify the anatomical scan range based on the physical characteristic of the patient.

In an aspect, a method for positioning a patient can include detecting a patient on a table proximate to the system, wherein the system is an x-ray imaging system, a magnetic resonance imaging (MRI) system, a positron emission tomography (PET) imaging system, a single-photon emission computed tomography (SPECT) imaging system, or a combination thereof. The method can also include detecting an anatomical scan range of the patient for acquisition in a medical image and determining that a first patient position prevents acquiring the medical image within the anatomical scan range. The method can also include generating a position indicator to provide to the patient, the position indicator representing a second patient position that allows the system to acquire the medical image within the anatomical scan range of the patient.

In another aspect, a non-transitory machine-readable medium for positioning a patient includes a plurality of instructions that, in response to execution by a processor, can cause the processor to detect a patient on a table proximate to the system. The plurality of instructions can also cause the processor to provide a position indicator to the patient using one or more lights of the system, a camera of the system, a removable sheet, a display device of the system, or a combination thereof, and provide a modified position indicator in response to input received by the system.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present techniques will be better understood from reading the following description of non-limiting examples, with reference to the attached drawings, wherein below:

FIG. 1 shows a pictorial view of an example imaging system;

FIG. 2 shows a block schematic diagram of an example imaging system;

FIG. 3 shows a process flow diagram illustrating an example method for providing position indicators to a patient proximate an imaging system, according to examples described herein;

FIG. 4 shows a process flow diagram illustrating an example method for providing position indicators to a patient proximate an imaging system, according to examples described herein;

FIGS. 5A and 5B show an example technique for providing a position indicator to a patient proximate an imaging system, according to examples described herein;

FIG. 6 shows an example technique for providing a position indicator to a patient proximate an imaging system, according to examples described herein;

FIG. 7 shows an example technique for providing a position indicator to a patient proximate an imaging system, according to examples described herein; and

FIG. 8 shows an example non-transitory, machine-readable media for providing position indicators to patients proximate imaging systems, according to examples described herein.

### DETAILED DESCRIPTION

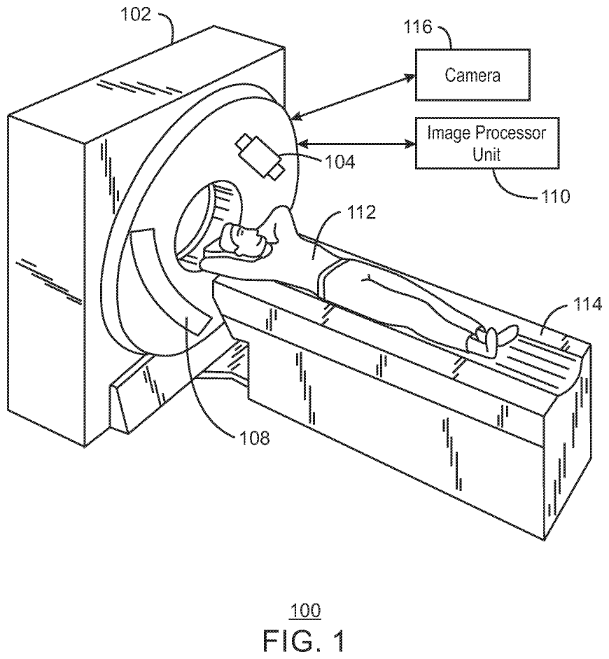

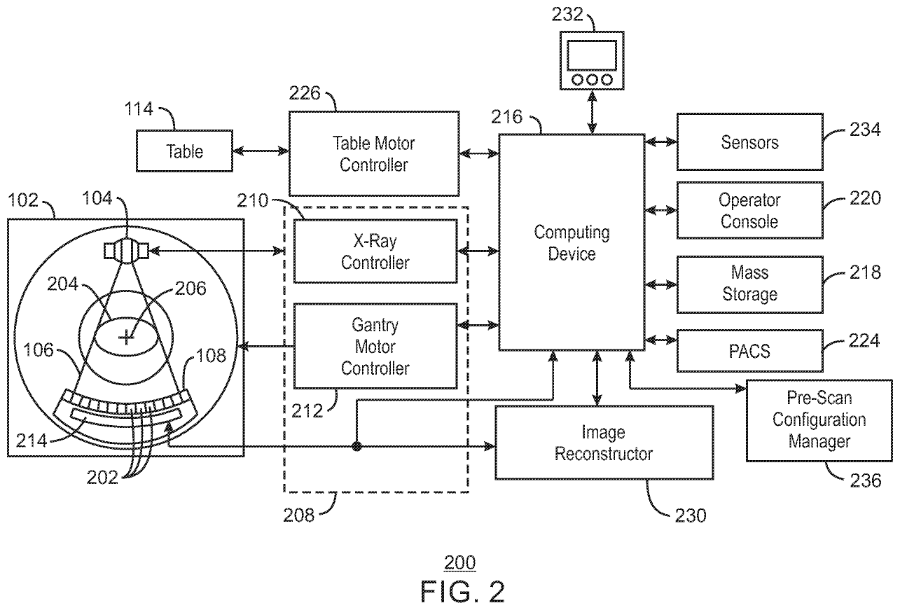

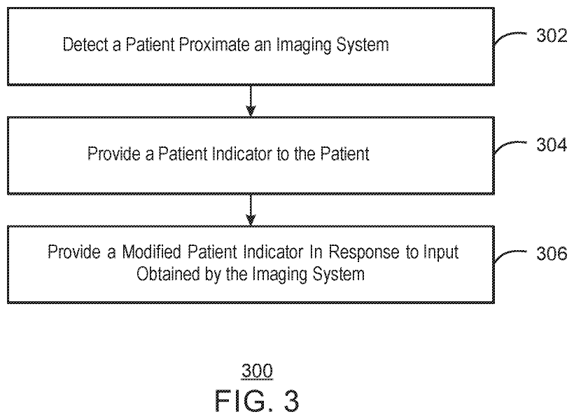

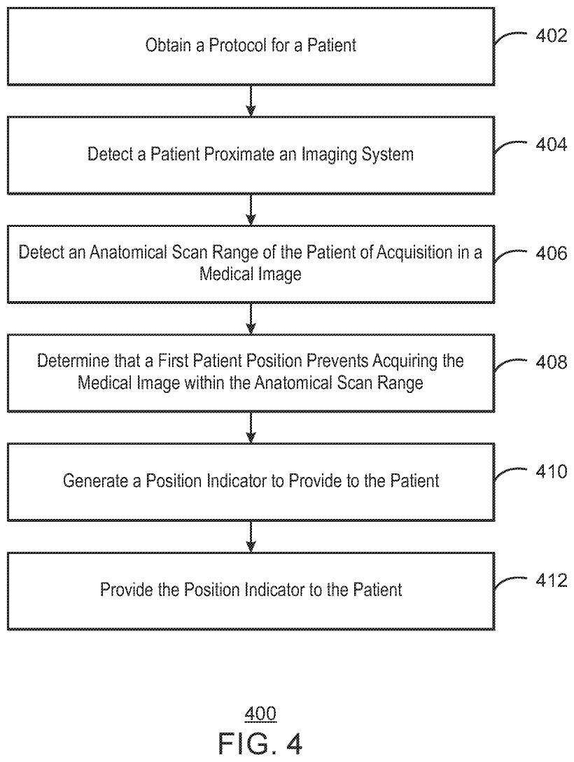

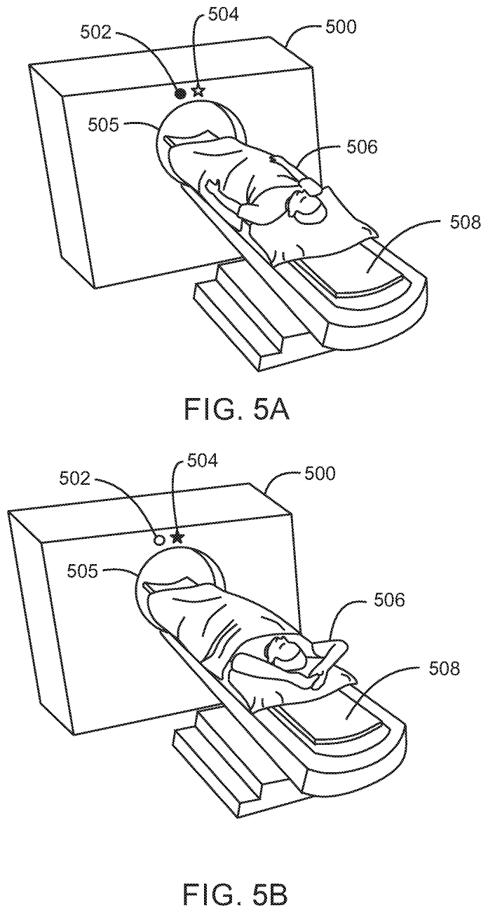

Embodiments of the present disclosure will now be described, by way of example, with reference to FIGS. 1-8, in which the following description relates to various examples of medical imaging systems. In particular, systems and methods are provided for capturing medical images in response to providing patient position indicators. An example of an imaging system that may be used to acquire images processed in accordance with the present techniques is provided in FIGS. 1 and 2. One approach to detecting a patient's position, such as the methods depicted in FIGS. 3 and 4, may include detecting a patient's position in relation to an imaging system and imaging components and providing a visual indicator to the patient representing where the patient is expected to be positioned on a table. FIGS. 5A, 5B, 6, and 7 show examples for providing indicators to patients prior to capturing a CT image of the patient. FIG. 8 shows an example non-transitory, machine-readable medium for providing indicators to a patient in response to detecting a position of the patient.

The technical effect of providing position indicators to a patient during a pre-scan configuration of a medical device can enable patients to position themselves without contact from clinicians. In some examples, a system can acquire medical images from one or more scan ranges with the patient in a requested patient position for each of the scan ranges. Accordingly, the present techniques have a technical advantage of providing position indicators to patients to acquire medical images with limited clinician contact, which can prevent the spread of highly transmissible diseases. The present techniques can also reduce the data storage and processing time of a medical imaging system by determining if a patient is in a requested position for a scan range prior to acquiring the medical images within the scan range. This can reduce processing time and data storage for medical images acquired for a patient in a position that cannot be analyzed.

Though a CT imaging system is described by way of example, it should be understood that the present techniques may also be useful when applied to images acquired using other imaging modalities, such as an x-ray imaging system, a magnetic resonance imaging (MRI) system, a positron emission tomography (PET) imaging system, a single-photon emission computed tomography (SPECT) imaging system, and combinations thereof (e.g., multi-modality imaging systems, such as PET/CT, PET/MR, or SPECT/CT imaging systems). The present discussion of a CT imaging modality is provided merely as an example of one suitable imaging modality.

FIG. 1 illustrates an example CT imaging system 100 configured for CT imaging. Particularly, the CT imaging system 100 is configured to image a subject 112 such as a patient, an inanimate object, one or more manufactured parts, and/or foreign objects such as implants, and/or contrast agents present within the body. In one embodiment, the CT imaging system 100 includes a gantry 102, which in turn, may further include at least one x-ray source 104 configured to project a beam of x-ray radiation 106 (see FIG. 2) for use in imaging the subject 112 laying on a table 114. Specifically, the x-ray source 104 is configured to project the x-ray radiation beams 106 towards a detector array 108 positioned on the opposite side of the gantry 102. Although FIG. 1 depicts only a single x-ray source 104, in certain embodiments, multiple x-ray sources and detectors may be employed to project a plurality of x-ray radiation beams 106 for acquiring projection data at different energy levels corresponding to the patient. In some embodiments, the x-ray source 104 may enable dual-energy gemstone spectral imaging (GSI) by rapid peak kilovoltage (kVp) switching. In some embodiments, the x-ray detector employed is a photon-counting detector which is capable of differentiating x-ray photons of different energies. In other embodiments, two sets of x-ray sources and detectors are used to generate dual-energy projections, with one set at low-kVp and the other at high-kVp. It should thus be appreciated that the methods described herein may be implemented with single energy acquisition techniques as well as dual energy acquisition techniques.

In certain embodiments, the CT imaging system 100 further includes an image processor unit 110 configured to identify the subject 112 on the table 114 and determine if a position of the subject 112 enables the CT imaging system 100 to acquire an image of a target volume of the subject 112. For example, the image processor unit 110 can capture camera images from a camera 116 coupled to the CT imaging system 100. The image processor unit 110 can analyze the camera images to determine a position of the subject 112 in relation to the table 114. In some examples, the CT imaging system 100 can also generate position indicators to provide to the subject 112 to indicate if the subject 112 is to move from a first position to a second position to enable acquiring the image of the target volume of the subject 112. In some examples, the camera 116 can project a position indicator onto the table 114, wherein the position indicator provides an outline for the arms, legs, head, or abdomen, of the subject 112. The position indicators are described in greater detail below in relation to FIGS. 2-7.

In some examples, the image processor unit 110 can determine if a patient is in an expected or requested position to acquire a target volume prior to acquiring an initialization image, following the acquisition of an initialization image, or following the acquisition of a diagnostic medical image. For example, the image processor unit 110 can detect if a patient is in a position to acquire a target volume representing a scan range of a subject 112 prior to acquisition of an initialization image, such as a scout image. The initialization image can be any image that uses a low dosage to capture an initial image for configuring the CT system 102, the placement of the subject 112 on the table 114, and the like. In some examples, the image processor unit 110 can determine if a patient is in a position to acquire a target volume within a scan range following the acquisition of the initialization image. As discussed below in relation to FIGS. 2-7, position indicators can be provided to the patient prior to acquiring an initialization image, following the acquisition of an initialization image, or any other suitable time. In some examples, initialization images can be acquired between one or more series of diagnostic scans of a subject 112. For example, the image processor unit 110 can acquire an initialization image following the acquisition of medical images for a protocol or a scan range. In some examples, the image processor unit 110 can capture or acquire any number of initialization images in any suitable sequence. In one example, the image processor unit 110 can acquire any number of consecutive initialization images until a patient or subject 112 is in a requested position. The image processor unit 110 can provide or display position indicators at any suitable time in response to detecting a position of a subject 112 on the table 114 prevents acquisition of a target volume.

In some examples, the image processor unit 110 can also reconstruct images of a target volume of the subject 112 using an iterative or analytic image reconstruction method. For example, the image processor unit 110 may use an analytic image reconstruction approach such as filtered back projection (FBP) to reconstruct images of a target volume of the patient. As another example, the image processor unit 110 may use an iterative image reconstruction approach such as advanced statistical iterative reconstruction (ASIR), conjugate gradient (CG), maximum likelihood expectation maximization (MLEM), model-based iterative reconstruction (MBIR), and so on to reconstruct images of a target volume of the subject 112. As described further herein, in some examples the image processor unit 110 may use both an analytic image reconstruction approach such as FBP in addition to an iterative image reconstruction approach.

In some CT imaging system configurations, an x-ray source projects a cone-shaped x-ray radiation beam which is collimated to lie within an X-Y-Z plane of a Cartesian coordinate system and generally referred to as an “imaging plane.” The x-ray radiation beam passes through an object being imaged, such as the patient or subject. The x-ray radiation beam, after being attenuated by the object, impinges upon an array of detector elements. The intensity of the attenuated x-ray radiation beam received at the detector array is dependent upon the attenuation of a radiation beam by the object. Each detector element of the array produces a separate electrical signal that is a measurement of the x-ray beam attenuation at the detector location. The attenuation measurements from all the detector elements are acquired separately to produce a transmission profile.

In some CT imaging systems, the x-ray source and the detector array are rotated with a gantry within the imaging plane and around the object to be imaged such that an angle at which the radiation beam intersects the object constantly changes. A group of x-ray radiation attenuation measurements, e.g., projection data, from the detector array at one gantry angle is referred to as a “view.” A “scan” of the object includes a set of views made at different gantry angles, or view angles, during one revolution of the x-ray source and detector. It is contemplated that the benefits of the methods described herein accrue to medical imaging modalities other than CT, so as used herein the term “view” is not limited to the use as described above with respect to projection data from one gantry angle. The term “view” is used to mean one data acquisition whenever there are multiple data acquisitions from different angles, whether from a CT, positron emission tomography (PET), or single-photon emission CT (SPECT) acquisition, and/or any other modality including modalities yet to be developed as well as combinations thereof in fused embodiments.

The projection data is processed to reconstruct an image that corresponds to a two-dimensional slice taken through the object or, in some examples where the projection data includes multiple views or scans, a three-dimensional rendering of the object. One method for reconstructing an image from a set of projection data is referred to in the art as the filtered back projection technique. Transmission and emission tomography reconstruction techniques also include statistical iterative methods such as maximum likelihood expectation maximization (MLEM) and ordered-subsets expectation-reconstruction techniques as well as iterative reconstruction techniques. This process converts the attenuation measurements from a scan into integers called “CT numbers” or “Hounsfield units,” which are used to control the brightness of a corresponding pixel on a display device.

In an “axial” scan, a CT table with the patient positioned thereon may be moved to the desired location and then maintained stationary while the x-ray beam is rotated within the gantry, collecting data. A plurality of measurements from slices of a target volume may be reconstructed to form an image of the entire volume.

To reduce the total scan time, a “helical” scan may be performed. To perform a “helical” scan, the patient is moved while the data for the prescribed number of slices is acquired. Such a system generates a single helix from a cone beam helical scan. The helix mapped out by the cone beam yields projection data from which images in each prescribed slice may be reconstructed.

As used herein, the phrase “reconstructing an image” is not intended to exclude examples of the present techniques in which data representing an image is generated but a viewable image is not. Therefore, as used herein, the term “image” broadly refers to both viewable images and data representing a viewable image. However, many embodiments generate (or are configured to generate) at least one viewable image.

FIG. 2 illustrates an example imaging system 200. In accordance with aspects of the present disclosure, the imaging system 200 is configured for imaging a patient or subject 204 (e.g., the subject 112 of FIG. 1). In one embodiment, the imaging system 200 includes the detector array 108 (see FIG. 1). The detector array 108 further includes a plurality of detector elements 202 that together sense the x-ray radiation beam 106 (see FIG. 2) that pass through the subject 204 (such as a patient) to acquire corresponding projection data. Accordingly, in one embodiment, the detector array 108 is fabricated in a multi-slice configuration including the plurality of rows of cells or detector elements 202. In such a configuration, one or more additional rows of the detector elements 202 are arranged in a parallel configuration for acquiring the projection data.

In certain embodiments, the imaging system 200 is configured to traverse different angular positions around the subject 204 for acquiring desired projection data. Accordingly, the gantry 102 and the components mounted thereon may be configured to rotate about a center of rotation 206 for acquiring the projection data, for example, at different energy levels. Alternatively, in embodiments where a projection angle relative to the subject 204 varies as a function of time, the mounted components may be configured to move along a general curve rather than along a segment of a circle.

As the x-ray source 104 and the detector array 108 rotate, the detector array 108 collects data of the attenuated x-ray beams. The data collected by the detector array 108 undergoes pre-processing and calibration to condition the data to represent the line integrals of the attenuation coefficients of the scanned subject 204. The processed data are commonly called projections.

In some examples, the individual detectors or detector elements 202 of the detector array 108 may include photon-counting detectors which register the interactions of individual photons into one or more energy bins. It should be appreciated that the methods described herein may also be implemented with energy-integrating detectors.

The acquired sets of projection data may be used for basis material decomposition (BMD). During BMD, the measured projections are converted to a set of material-density projections. The material-density projections may be reconstructed to form a pair or a set of material-density map or image of each respective basis material, such as bone, soft tissue, and/or contrast agent maps. The density maps or images may be, in turn, associated to form a volume rendering of the basis material, for example, bone, soft tissue, and/or contrast agent, in the imaged volume.

Once reconstructed, the basis material image produced by the imaging system 200 reveals internal features of the subject 204, expressed in the densities of two basis materials. The density image may be displayed to show these features. In traditional approaches to diagnosis of medical conditions, such as disease states, and more generally of medical events, a radiologist or physician would consider a hard copy or display of the density image to discern characteristic features of interest. Such features might include lesions, sizes and shapes of particular anatomies or organs, and other features that would be discernable in the image based upon the skill and knowledge of the individual practitioner.

In one embodiment, the imaging system 200 includes a control mechanism 208 to control movement of the components such as rotation of the gantry 102 and the operation of the x-ray source 104. In certain embodiments, the control mechanism 208 further includes an x-ray controller 210 configured to provide power and timing signals to the x-ray source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. The DAS 214 may be further configured to selectively aggregate analog data from a subset of the detector elements 202 into so-called macro-detectors, as described further herein. The data sampled and digitized by the DAS 214 is transmitted to a computer or computing device 216. In one example, the computing device 216 stores the data in a storage device or mass storage 218. The storage device 218, for example, may include a hard disk drive, a floppy disk drive, a compact disk-read/write (CD-R/W) drive, a Digital Versatile Disc (DVD) drive, a flash drive, and/or a solid-state storage drive.

Additionally, the computing device 216 provides commands and parameters to one or more of the DAS 214, the x-ray controller 210, and the gantry motor controller 212 for controlling system operations such as data acquisition and/or processing. In certain embodiments, the computing device 216 controls system operations based on operator input. The computing device 216 receives the operator input, for example, including commands and/or scanning parameters via an operator console 220 operatively coupled to the computing device 216. The operator console 220 may include a keyboard (not shown) or a touchscreen to allow the operator to specify the commands and/or scanning parameters.

Although FIG. 2 illustrates only one operator console 220, more than one operator console may be coupled to the imaging system 200, for example, for inputting or outputting system parameters, requesting examinations, plotting data, and/or viewing images. Further, in certain embodiments, the imaging system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks, wireless telephone networks, wireless local area networks, wired local area networks, wireless wide area networks, wired wide area networks, etc.

In one embodiment, for example, the imaging system 200 either includes, or is coupled to, a picture archiving and communications system (PACS) 224. In an example implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-defined commands and parameters to operate a table motor controller 226, which in turn, may control a table 114 which may be a motorized table. Specifically, the table motor controller 226 may move the table 114 for appropriately positioning the subject 204 in the gantry 102 for acquiring projection data corresponding to the target volume of the subject 204.

As previously noted, the DAS 214 samples and digitizes the projection data acquired by the detector elements 202. Subsequently, an image reconstructor 230 uses the sampled and digitized x-ray data to perform high-speed reconstruction. Although FIG. 2 illustrates the image reconstructor 230 as a separate entity, in certain embodiments, the image reconstructor 230 may form part of the computing device 216. Alternatively, the image reconstructor 230 may be absent from the imaging system 200 and instead the computing device 216 may perform one or more functions of the image reconstructor 230. Moreover, the image reconstructor 230 may be located locally or remotely, and may be operatively connected to the imaging system 200 using a wired or wireless network. In some examples, computing resources in a “cloud” network cluster can be used for the image reconstructor 230.

In one embodiment, the image reconstructor 230 stores the images reconstructed in the storage device 218. Alternatively, the image reconstructor 230 may transmit the reconstructed images to the computing device 216 for generating useful patient information for diagnosis and evaluation. In certain embodiments, the computing device 216 may transmit the reconstructed images and/or the patient information to a display or display device 232 communicatively coupled to the computing device 216 and/or the image reconstructor 230. In some embodiments, the reconstructed images may be transmitted from the computing device 216 or the image reconstructor 230 to the storage device 218 for short-term or long-term storage.

In some examples, the imaging system 200 can implement a pre-scan configuration prior to acquiring diagnostic medical images for the image reconstructor 230. For example, the pre-scan configuration can include a graphical user interface provided to the display device 232 of the imaging system 200. The graphical user interface displayed by the display device 232 can provide a live video stream of a patient on a table 114 of the imaging system 200.

In some examples, the storage device 218 can include one or more applications that determine data related to a patient' position based at least in part on sensor data from sensors 234. The sensors 234 can include, in some examples, a gyroscope, an accelerometer, an ambient light sensor, a camera, and the like. The sensors 234 can receive or capture sensor data that can include camera images, pressure sensor data, or any other sensor data, that indicates a position of a patient on the table 114 of the imaging system 200. As discussed in greater detail below in relation to FIG. 3, the sensor data can be analyzed and aggregate to detect or determine a position of a patient or subject 112 on a table 114. In some examples, the sensors 234 can be electronically coupled to the computing device 216 or the sensors 234 can be coupled to the CT system 102 and the computing device 216 can receive the sensor data from the CT system 102 with any suitable wired or wireless interface. In some examples, the sensors 234 can detect sensor data for a table 114 that can be either vertically positioning or horizontally positioned proximate the imaging system 200.

In some examples, the computing device 216, the CT system 102, or any combination thereof, can execute instructions received or generated by a pre-scan configuration manager 236. The pre-scan configuration manager 236 can be stored in the mass storage 218, in memory (not depicted) of the computing device 216, in memory (not depicted) of the CT system 102, or in any suitable storage device or memory device coupled to the CT system 102. In some examples, the pre-scan configuration manager 236 can implement the pre-scan configuration by generating instructions for providing one or more patient position indicators to the subject 112. For example, the pre-scan configuration manager 236 can analyze and compare the position of a subject 112 to a target volume for a diagnostic medical scan. If the data related to the patient's position indicates that the subject 112 is not in the requested position for acquiring a medical image, the pre-scan configuration manager 236, using the computing device 216, the CT system 102, or any combination thereof, can provide any number of indicators to help the subject 112 move or change positions to become aligned on the table 114 of the imaging system 200. The position indicators can include any number of lights, audio messages, projections, and the like. In some examples, the computing device 216 can generate the position indicators and transmit instructions to the CT system 102 to provide the position indicators to the subject 112.

In some examples, the display 232 coupled to the computing device 216 enables an operator or clinician to access or view data from the pre-scan configuration manager 236 and to evaluate the imaged anatomy. The display 232 may also allow the operator to select a volume of interest (VOI) and/or request patient information, for example, via a graphical user interface (GUI) for a subsequent scan or processing. In some examples, the display 232 can be electronically coupled to the computing device 216, the CT system 102, or any combination thereof. For example, the display 232 can receive data, such as position indicators, from the pre-scan configuration manager 236, and provide the position indicators to a subject 112 proximate the CT system 102 by displaying the position indicators on the display 232. In some examples, the display 232 can display or provide the position indicators to clinicians or operators proximate the computing device 216. The computing device 216 may be located proximate the CT system 102 or the computing device 216 may be located in another room, area, or a remote location.

In some examples, the pre-scan configuration manager 236 can be partially, or entirely, implemented in hardware of the CT system 102, the computing device 216, or any combination thereof. For example, the functionality of the pre-scan configuration manager 236 can be implemented with an application specific integrated circuit, logic implemented in an embedded controller, or in logic implemented in a processor, among others. In some examples, the functionality of the pre-scan configuration manager 236 can be implemented with logic, wherein the logic, as referred to herein, includes any suitable hardware (e.g. a processor, a graphics card, or the like), software (e.g. an application, an operating system, or the like), firmware, or any suitable combination of hardware, software, and firmware.

The various methods and processes (such as the methods described below with reference to FIG. 3) described further herein may be stored as executable instructions in non-transitory memory on a computing device (or controller) in imaging system 200. In one embodiment, image reconstructor 230 and the pre-scan configuration manager 236 may include such executable instructions in non-transitory memory, and may apply the methods described herein to provide patient indicators. In another embodiment, computing device 216 may include the instructions in non-transitory memory, and may apply the methods described herein, at least in part, to position a patient proximate the imaging system 200. In yet another embodiment, the methods and processes described herein may be distributed across the CT system 102 and the computing device 216.

FIG. 3 illustrates an example process flow diagram for detecting a patient's position. In some examples, the method 300 can be implemented with any suitable device such as the CT system 100 of FIG. 1 or the imaging device 200 of FIG. 2.

At block 302, the method 300 includes detecting or identifying a patient proximate to the imaging system. For example, the patient can be detected on a table coupled to the imaging system or adjacent to the imaging system. In some examples, the method can include detecting a patient on a table using any suitable number of images from a camera, sensor data from any number of sensors, or a combination thereof. For example, the sensor data can be detected or obtained from pressure sensors, gyroscopes, accelerometers, compasses, and the like. In some examples, images of a table or sensor data collected from sensors within the table or proximate to the table can be analyzed to determine if a patient is residing on a table. Techniques for detecting a patient proximate an imaging system are described in greater detail below in relation to block 404 of FIG. 4.

At block 304, the method 300 can include providing a position indicator to the patient using one or more lights of the system, a camera of the system, a removable sheet, a display device of the system, or a combination thereof. For example, the method 300 can include projecting any number of lights onto a table of an imaging system, wherein the lights indicate if a patient is properly aligned on the table for a medical image to be acquired. In some examples, the lights can be red, green, or any other suitable color to indicate if a patient is in an expected position to acquire a medical image. The lights can be projected from a camera, from lights within a bore of an imaging system, or with any other suitable device.

In some examples, a removable sheet can be affixed to a table of an imaging system to indicate an expected position of a patient. For example, a representation of the arms of a patient may be outlined on the sheet to indicate if a patient's arms should be raised above the patient's head or if the patient's arms should remain by the abdomen of the patient. In some examples, the removable sheet can include a representation of any suitable region or area of a patient in order to provide an expected position of the patient.

In some examples, a display system coupled to the imaging system can provide a patient indicator representing an expected position of a patient. For example, the display system can provide an outline of the expected position of the patient on an empty table. In some examples, the display system can provide a real-time video stream captured by a camera coupled to the imaging system. The display system may overlay or combine the real-time video stream and a position indicator indicating the expected position of the patient. For example, the display system can provide a representation of an expected position of a patient with a solid line, a dotted line, or any other suitable representation that is displayed along with the real-time video stream. A patient can change the position of the patient's arms, alignment on the table, and the like, so that the patient's image captured in the real-time video stream is within the representation of the expected position.

At block 306, the method 300 can include providing a modified position indicator in response to input received by the system. In some examples, the modified position indicator can be a different color projected light than the position indicator, a representation provided by a display system, or the like. The modified position indicator can provide feedback to a patient and represent that a patient is in an expected position for acquiring a medical image. For example, the modified position indicator may be a green light that is projected onto a table in response to a patient moving into an expected position for acquisition of a medical image. In some examples, the modified position indicator can be any suitable audio message, visual image, or a combination thereof. For example, the modified position indicator can include input such as an audio message provided by the imaging system to the patient to indicate how the patient should be repositioned. The input, which can include an audio message or a visual image, among others, can be received, obtained, or otherwise acquired from a technologist operating an imaging device or any other suitable source. The input can indicate a patient's current unexpected position, an expected position of a patient, directions related to transitioning the patient from the unexpected position to the expected position, and the like. A position indicator representing an unexpected position of a patient and a modified position indicator representing an expected position of the patient are described in greater detail below in relation to FIGS. 5A and 5B.

The process flow diagram of method 300 of FIG. 3 is not intended to indicate that all of the operations of blocks 302-306 of the method 300 are to be included in every example. Additionally, the process flow diagram of method 300 of FIG. 3 describes a possible order of executing operations. However, it is to be understood that the operations of the method 300 can be implemented in various orders or sequences. In addition, in some examples, the method 300 can also include fewer or additional operations.

FIG. 4 illustrates an example process flow diagram for detecting a patient's position. In some examples, the method 400 can be implemented with any suitable device such as the CT system 100 of FIG. 1 or the imaging device 200 of FIG. 2.

At block 402, the method 400 includes receiving, detecting, or otherwise obtaining a protocol for a patient. The protocol, as referred to herein, indicates a single medical image to capture or a series of medical images to capture. In some examples, the protocol can indicate a scan range, a region of the body corresponding to the scan range, a dosage amount, and the like. The scan range can indicate a starting location and an end location for each medical image to be captured by the CT device. In some examples, a protocol can be shared between multiple patients or each patient can have an individualized protocol. For example, an individualized protocol can specify a scan range based on a height of a patient or a weight of a patient.

At block 404, the method 400 can include detecting or identifying a patient proximate to the imaging system. In some examples, the method can include detecting any suitable number of images from a camera, sensor data from any number of sensors, or a combination thereof. For example, the method 400 can include detecting the patient proximate the system by monitoring a table proximate the system with a continuous set of camera images provided to a machine learning algorithm. In some examples, the machine learning algorithm can analyze or monitor the camera images and determine if an object is residing on the table of the imaging system. The machine learning techniques can also, in some examples, determine if the detected object on a table is a subject 112 corresponding to a target volume to be acquired. For example, the machine learning technique may be initialized with images of various objects and subjects, such as patients, so that the machine learning technique can distinguish between patients and additional objects that may be placed on the table of the imaging system.

In some examples, the table of the imaging system can include any number of sensors such as pressure sensors, gyroscopes, accelerometers, compasses, and the like. The sensor data collected from the sensor can be used alone or in combination with the camera images to determine if a patient resides on the table of the imaging system. For example, the gyroscope or pressure sensors can determine a weight, a size, or both a weight and size of an object placed on the table. In some examples, objects that exceed a predetermined threshold can be identified as a patient. For example, objects that exceed a first threshold but do not exceed a second threshold can be identified as pediatric patients and objects that exceed both the first threshold and the second threshold can be identified as adult patients. The first threshold can be any suitable weight, such as 30 pounds, 40 pounds, 50 pounds, or the like. In some examples, the second threshold can be any suitable weight such as 100 pounds, 120 pounds, 130 pounds, or the like. In some examples, the first threshold and the second threshold can also represent a portion of the table that is covered by the patient such that a smaller portion of the table being covered can represent a pediatric patient and a larger portion of the table being covered can represent an adult patient. The area of the table being covered by the patient can be detected by a series of pressure sensors within or proximate to the table of the imaging system. The area of the table being covered can also be detected by the portion of the table obscured by a patient in a camera image or by a number of ambient light sensors placed proximate the table that detect a change in light. In some examples, the method 400 can include detecting a patient residing on a table of a medical imaging device based on sensor data that can include pressure sensor data from the table, ambient light sensor data to detect that an object has blocked a set of light sources, gyroscope data to indicate a position of a table has shifted due to an object residing on the table, or the like.

At block 406, the method 400 can include detecting, receiving, or otherwise obtaining an anatomical scan range of the patient for acquisition in a medical image. In some examples, the anatomical scan range can indicate a starting point and an end point for acquiring image data by the imaging system. For example, the anatomical scan range can indicate any number of inches, centimeters, feet, meters, or the like, to be scanned by the image system. In some examples, the anatomical scan range can be specified in a protocol indicating one or more diagnostic scans to be performed for a number of areas of a patient's body. For example, an anatomical scan can represent a range to be scanned for a head scan, a chest scan, an abdomen scan, and the like. In some examples, the method 400 can include detecting a different size of anatomical scan ranges based on whether the imaging system detects a pediatric patient or an adult patient. For example, an anatomical scan range can be adjusted or rescaled to a smaller size if a pediatric patient is detected such that a smaller head scan region, abdomen scan region, or the like, is used to acquire the diagnostic medical images. In some examples, the patient indicator can also be adjusted to provide a larger or smaller outline based on a size of the subject or patient.

At block 408, the method 400 can include determining that a first patient position prevents acquiring the medical image within the anatomical scan range. In some examples, the method can include comparing a shape of a patient on the table of the imaging device to a predetermined configuration of the patient within the anatomical scan range. For example, the method 400 can include using any suitable machine learning techniques to compare an outline of a head in relation to a head holder, an outline of an abdomen in relation to a predefined area of the table for abdomen scans, and the like. In some examples, the machine learning technique can identify and detect if a patient is in a first position that prevents acquiring medical imaging data from the anatomical scan range. For example, the head of the patient may not be placed on a head holder attached to the imaging system, a patient may be positioned too low or too high on a table of the imagining system or within a foot extender coupled to the imaging system, or the like.

In some examples, the method 400 can include determining that the first patient position is not aligned or positioned properly in relation to any number of components attached or coupled to the imaging system. The components of the system can include a table, a tilted head holder, a flat head holder, a foot extender, a knee pad support device, electrodes, child positioning equipment, a chin strap, a table pad, a cradle overlay, or a combination thereof. In some examples, any number of the components can be used to capture a medical image or a series of medical images of a patient. For example, a head holder and a knee support device may be used for capturing a full body scan image of a patient.

At block 410, the method can include generating a position indicator to provide to the patient. The position indicator can represent a second patient position that allows the system to acquire the medical image within the anatomical scan range of the patient. In some examples, the position indicator can be presented to a patient as any number of lights within a table of the imaging system, by a series of lights projected from within the bore hole of the imaging system, from an image projected onto the table from a camera, or the like. For example, the position indicators can provide an indication to the patient that the first patient position is incorrect and an indication that the patient has modified the position of the patient to a correct position. In some examples, the position indicators can provide one or more red lights for the incorrect first patient position and one or more green lights for the correct second patient position. In some examples, any suitable different colors can be used to represent the first indication and the second indication. The position indicators can also include any number of different shapes, images, or the like, that indicate to a patient if the position of the patient enables acquiring diagnostic medical images of a target volume.

At block 412, the method 400 can include providing the position indicator to the patient. In some examples, the position indicator can be provided using any suitable number of lights, sounds, materials placed on the table of the imaging device, or the like. For example, an audio message can indicate a direction and distance for the patient to move, or provide instructions regarding how to interpret or use additional light indicators. In some examples, an audio message can provide a distance for the patient to move in one or more directions until the system detects that the patient is in a predetermined patient position.

The position indicators can also include any number of lights arranged or configured proximate to the table or the imaging system, among others. For example, the lights can be arranged in any suitable pattern that enables a patient to determine when the patient's position enables acquisition of a target volume. In some examples, the lights can be lined along the table of an imaging device, the lights can be placed along the CT system, or the lights can be placed in any suitable location proximate the table or CT system. Each light may represent an area of the table and the light can provide or indicate a first indication that the patient's position within the area of the table is expected and a second indication that the patient's position within the area is incorrect and prevent acquisition of a target volume.

In some examples, a preconfigured removable sheet that provides an outline of the second patient position can be coupled to the table of an imaging system. For example, a sheet of paper or any other suitable material can be coupled or otherwise attached to the table. The paper or material representing the second patient position can include an adhesive material that maintains a static or constant location of the removable sheet of paper on the table of the imaging device and provides an outline within the anatomical scan range for the expected or predetermined patient position. For example, the sheet of paper, or any other material, can indicate that an abdomen of the patient is to be placed within a predetermined area of the table represented by an outline of the sheet of paper. In some examples, the removable sheet of paper, or any other suitable material indicating the second patient position, may not have any adhesive, or the removable sheet of paper may be textured to provide friction to prevent the removable sheet of paper from moving out of place on the table of the imaging system.

In some examples, the position indicator can enable the patient to be positioned as expected for acquiring a target volume and an initialization image may not be acquired. The position indicator can also prevent the acquisition of diagnostic medical images with incomplete target volume areas. For example, the position indicator can ensure that the diagnostic images acquired for a target volume include the target volume due to the patient being in the predetermined position on the table. The position indicator can also enable the patient to modify or change the position of the patient prior to acquiring the initialization image and the diagnostic medical images of a predetermined protocol.

The process flow diagram of method 400 of FIG. 4 is not intended to indicate that all of the operations of blocks 402-412 of the method 400 are to be included in every example. Additionally, the process flow diagram of method 400 of FIG. 4 describes a possible order of executing operations. However, it is to be understood that the operations of the method 400 can be implemented in various orders or sequences. In addition, in some examples, the method 400 can also include fewer or additional operations. In some examples, the method 400 can include detecting a physical characteristic of the patient. The physical characteristic can include a height of the patient, a weight of the patient, or a combination thereof. The method 400 can also include modifying the anatomical scan range based on the physical characteristic of the patient.

FIGS. 5A and 5B illustrate an example medical imaging device that provides position indicators. In the example medical imaging device 500 of FIG. 5A, lights 502 and 504 are located above the bore hole 505. In some examples, the lights 502 and 504 can have different shapes, as well as different colors, and any other suitable distinguishing characteristics. In some examples, the subject 506 may be positioned improperly so that a target volume of the subject 506 cannot be acquired. A light 502 can display an indication that the subject 506 is not residing on the table 508 in a position that enables capturing a diagnostic medical image of a target volume. In some examples, light 504 may not receive power or may provide a different color than light 502 until the subject 506 moves or adjusts the position of the subject 506 to an expected or requested position.

In FIG. 5B, the subject 506 has adjusted the position of the subject's 506 arms. The imaging system 500 can detect the adjusted position of the subject 506 and compare the adjusted position of the subject 506 to the expected position for acquiring a medical image of a target volume. In some examples, light 504 can display a notification or indicator, such as a modified position indicator, that the subject 506 is in an expected and requested position and light 502 can turn off to further indicate to the subject 506 that the adjusted position is correct.

In some examples, light 502 can display a light of a first color, such as red, among others, in response to detecting that the patient is in an incorrect position on a table proximate the medical imaging device 500. In some examples, light 504 can display a second color, such as green, among others, in response to detecting that the patient is in a correct position that enables acquiring imaging data within a predetermined anatomical scan range. For example, the light 504 can indicate to the patient to remain in a particular position until one or more series of scans have been acquired by the medical imaging system 500. In some examples, the lights 502 and 504 transition from illumination of light 502 to illumination of light 504 in response to the patient moving on the table until the patient is in the predetermined or expected position for acquiring imaging data.

In some examples, the lights 502 and 504 can change as imaging data is acquired in response to a patient shifting position from a predetermined and expected position to an improper position that prevents acquisition of a target volume. For example, the light 504 can be turned off and light 502 can be illuminated if a patient moves the patient's head to a position outside of a predetermined scan range. The lights 502 and 504 can transition to indicate that a patient is in an expected position or an unexpected position prior to acquiring an initialization image, after acquiring an initialization image and before acquiring a diagnostic medical image, or during the acquisition of the diagnostic medical image, among others.

It is to be understood that the imaging system 500 of FIGS. 5A and 5B is an example and that other configurations of the imaging system may include additional lights 502 and 504, fewer lights. The lights 502 and 504 can also be placed at different locations of the imaging system 500 such as on a table 508, on the sides of the bore hole 505, or any other suitable location. In some examples, the lights 502 and 504 can be placed in any location viewable by the subject or patient.

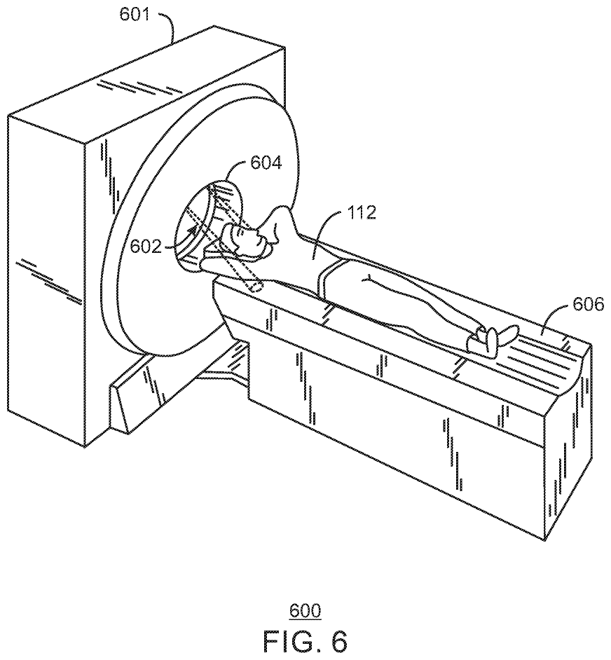

FIG. 6 is an example of a system providing a position indicator. In the imaging system 600, a CT system 601 can include one or more lights 602 can be projected from the bore hole 604 onto a table 606. The one or more lights 602 can indicate to the patient if the patient is residing on the table 606 in an incorrect position that prevents acquiring imaging data of a target volume. For example, the one or more lights 602 can project a green light if the patient is in the correct position or a red light if the patient is in an incorrect position. In some examples, the one or more lights 602 can be projected onto the table to indicate where a patient is to be positioned. For example, the one or more lights 602 may be movable so that the one or more lights 602 can project onto the table 606 where the patient is to be positioned. In some examples, the one or more lights 602 can project an outline onto the table 606 for the patient to lie within. The one or more lights 602 can also project lights onto the table proximate incorrect patient positions. For example, the one or more lights 602 can project a red light onto the table 606 proximate the patient if the patient is skewed to the side of the table 606. In some examples, the CT system 601 can project a position indicator onto the table 606, wherein the patient indicator comprises a configuration image representing the second patient position that enables the system to acquire the medical image within the anatomical scan range. For example, the CT system 601 may use the one or more lights 602 to project an outline of a configuration image onto the table 606 that indicates an expected position of a patient's head, arms, legs, abdomen, and the like.

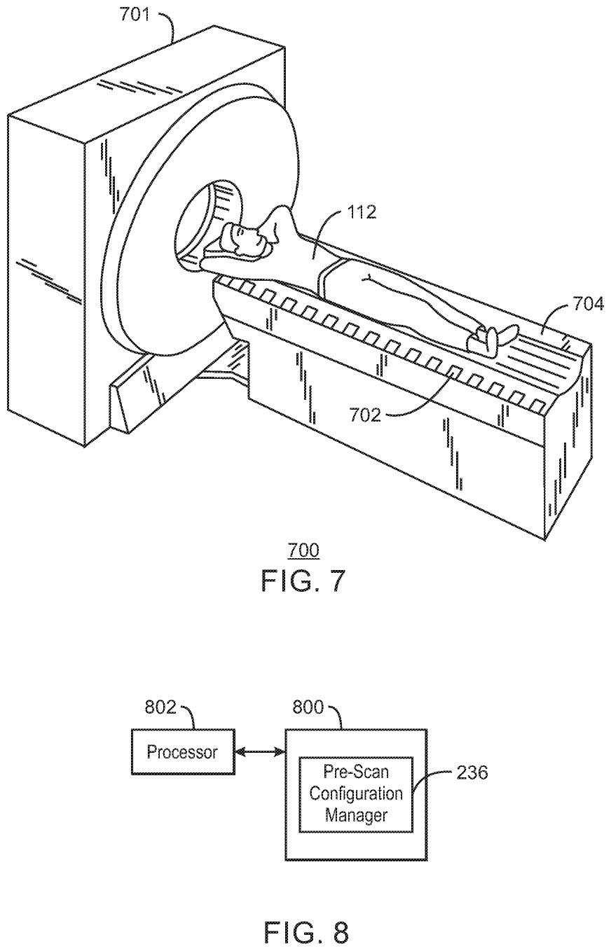

FIG. 7 is an example of a system providing a position indicator. In some examples imaging systems 700, can include a CT system 701 that includes lights 702 can be included in a table 704 of the imaging systems 700. In some examples, the lights 702 can be included in the table 704 in any suitable configuration or arrangement. For example, the lights 702 can be included in a row or a line along an edge of the table 704. If the patient is improperly positioned on the table 704, the lights 702 can change colors or provide any other suitable indication that the patient is to change positions. For example, the lights 702 proximate a scan range for a patient can change colors, flash brighter, blink, or the like, to indicate that the patient is to change positions in the scan range area of the table 704. In some examples, the lights 702 can be included on one or more sides of the CT system 701, represented within a digital display connected to the CT system 701, or the like. The lights 702 can also be included in one contiguous section that includes one or more lights 702. For example, a contiguous section or segment along one or more edges of the table 704 can include any number of lights 702 that provide a position indicator.

FIG. 8 is an example of a non-transitory machine-readable medium for detecting a position of a patient, in accordance with examples. The non-transitory, machine-readable medium 800 can implement the functionalities of the image processor unit 110 of FIG. 1 and the computing device 216 of FIG. 2, among others. For example, a processor 802 in a control system of a CT system 102, a computing device 216, or any other suitable device, can access the non-transitory, machine-readable media 800.

In some examples, the non-transitory, machine-readable medium 800 can include instructions to execute a pre-scan configuration manager 236. For example, the non-transitory, machine-readable medium 800 can include instructions for the pre-scan configuration manager 236 that cause the processor 802 to generate and provide position indicators to a subject proximate an imaging system. In some examples, the non-transitory, machine-readable medium 800 can include instructions to implement any combination of the techniques of the pre-scan configuration manager 236 described above.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A system for self-positioning a patient comprising:
a processor to:
detect a patient proximate to the system;
detect an anatomical scan range of the patient for acquisition in a medical image;
determine that a first patient position prevents acquisition of the medical image within the anatomical scan range; and
generate a position indicator to provide to the patient, the position indicator representing a second patient position that allows the system to acquire the medical image within the anatomical scan range of the patient.

2. The system of claim 1, wherein the system is an x-ray imaging system, a magnetic resonance imaging (MRI) system, a positron emission tomography (PET) imaging system, a single-photon emission computed tomography (SPECT) imaging system, or a combination thereof.

3. The system of claim 1, wherein the patient resides on a table proximate to the system, and wherein the position indicator comprises one or more lights displayed by the system using the table or using a display device of the system.

4. The system of claim 3, wherein the one or more lights comprise at least a first light displaying a first color representing the first patient position or a second color representing the second patient position.

5. The system of claim 3, wherein the processor is to project the position indicator onto the table, and wherein the patient indicator comprises a configuration image representing the second patient position that enables the system to acquire the medical image within the anatomical scan range.

6. The system of claim 5, wherein the system is to project the position indicator from within a bore hole of the system, wherein the position indicator comprises one or more projected lights representing the second position.

7. The system of claim 3, wherein the processor is to:
capture one or more camera images of the patient with a camera; and
determine the first patient position based on the one or more camera images.

8. The system of claim 7, wherein the processor is to execute a machine learning technique to identify the first patient position.

9. The system of claim 1, wherein the processor is to:
detect a size of the patient; and
adjust the position indicator based on the size of the patient.

10. The system of claim 3, wherein the system comprises a camera to project the position indicator onto the table.

11. The system of claim 1, wherein the position indicator comprises an audio message that provides a distance for the patient to move in one or more directions until the system detects that the patient is in the second patient position.

12. The system of claim 1, wherein the system further comprises a material coupled to the table, wherein the material provides the position indicator, the position indicator comprising an outline of the second patient position.

13. The system of claim 3, wherein the table is configured in a vertical position proximate the system or wherein the table is configured in a horizontal position proximate the system.

14. The system of claim 3, wherein the table comprises one or more lights that provide the patient indicator.

15. The system of claim 1, wherein the processor is to:
detect a physical characteristic of the patient, the physical characteristic comprising a height of the patient; and
modify the anatomical scan range based on the physical characteristic of the patient.

16. A method for self-positioning a patient comprising:
detecting a patient on a table proximate to the system, wherein the system is an x-ray imaging system, a magnetic resonance imaging (MRI) system, a positron emission tomography (PET) imaging system, a single-photon emission computed tomography (SPECT) imaging system, or a combination thereof;
detecting an anatomical scan range of the patient for acquisition in a medical image;
determining that a first patient position prevents acquiring the medical image within the anatomical scan range; and
generating a position indicator to provide to the patient, the position indicator representing a second patient position that allows the system to acquire the medical image within the anatomical scan range of the patient.

17. The method of claim 16, wherein the table is configured in a vertical position proximate the system or wherein the table is configured in a horizontal position proximate the system.

18. The method of claim 16, wherein the position indicator comprises one or more lights displayed by the system using the table or using a display device of the system.

19. The method of claim 18, wherein the one or more lights comprise at least a first light displaying a first color representing the first patient position or a second color representing the second patient position.

20. A non-transitory machine-readable medium for self-positioning a patient comprising a plurality of instructions that, in response to execution by a processor, cause the processor to:
detect a patient on a table proximate to a system;
provide a position indicator to the patient using one or more lights of the system, a camera of the system, a removable sheet, a display device of the system, or a combination thereof; and
provide a modified position indicator in response to input received by the system.
